(19) United States
(12) Patent Application Publication
(10) Pub. No.: US 2007/0232983 A1
(43) Pub. Date: Oct. 4, 2007

(54) Handheld apparatus to deliver active agents to biological interfaces

(76) Inventor: Gregory A. Smith, Union City, CA (US)

Correspondence Address: SEED INTELLECTUAL PROPERTY LAW GROUP PLLC, 701 FIFTH AVE, SUITE 5400, SEATTLE, WA, 98104, US

(21) Appl. No.: 11/514,296

(22) Filed: Aug. 30, 2006

Related U.S. Application Data
(60) Provisional application No. 60/722,759, filed on Sep. 30, 2005.

(51) Int. Cl. A61N 1/30

(52) U.S. Cl. 604/20

(57) ABSTRACT

A device to deliver an active agent to a biological entity includes a handle shaped portion configured to be grasped to provide a first portion of the biological entity, and a probe shaped portion extending from the handle shaped portion configured to be positioned on or proximate a second portion of the biological entity to complete a circuit path from a power source through the biological entity. The device may employ iontophoresis. The device may be configured to approximate the shape of a conventional toothbrush or electric tooth brush.

### CROSS-REFERENCE TO RELATED APPLICATION

This application claims the benefit under 35 U.S.C. §119(e) of U.S. Provisional Patent Application No. 60/722,759, filed Sep. 30, 2005.

### BACKGROUND OF THE INVENTION

1. Field of the Invention

This disclosure generally relates to the field of iontophoresis, and more particularly to the delivery of active agents such as therapeutic agents or drugs to a biological interface, for example skin, mucous membrane or tooth.

2. Description of the Related Art

Iontophoresis employs an electromotive force to transfer an active agent such as an ionic drug or other therapeutic agent to a biological interface, for example skin or mucus membrane.

Iontophoresis devices typically include an active electrode assembly and a counter electrode assembly, each coupled to opposite poles or terminals of a power source, for example a chemical battery. Each electrode assembly typically includes a respective electrode element to apply an electromotive force. Such electrode elements often comprise a sacrificial element or compound, for example silver or silver chloride.

The active agent may be either cation or anion, and the power source can be configured to apply the appropriate voltage polarity based on the polarity of the active agent. Iontophoresis may be advantageously used to enhance or control the delivery rate of the active agent. As discussed in U.S. Pat. No. 5,395,310, the active agent may be stored in a reservoir such as a cavity. Alternatively, the active agent may be stored in reservoir such as a porous structure or a gel. Also as discussed in U.S. Pat. No. 5,395,310, an ion exchange membrane may be positioned to serve as a polarity selective barrier between the active agent reservoir and the biological interface.

Commercial acceptance of iontophoresis devices is dependent on a variety of factors, such as cost to manufacture, shelf life or stability during storage, efficiency and/or timeliness of active agent delivery, biological capability and/or disposal issues. Commercial acceptance of iontophoresis devices is dependent on ease of use, and the ability to delivery active agent to precisely locations, in controlled quantities. An iontophoresis device that addresses one or more of these factors is desirable.

### BRIEF SUMMARY OF THE INVENTION

In one aspect, a device for delivering an active agent under the influence a power source to a biological entity includes: a handle sized and dimensioned to be grasped, a plurality of probes extending from the handle a counter electrode assembly at least partially positioned in the handle, the counter electrode assembly comprising at least a counter electrode element operable to supply an electrical potential of a first polarity, the counter electrode assembly operable to provide an electrically conductive path between a first biological interface and the counter electrode element when the handle is grasped, and at least one active electrode assembly positioned proximate the plurality of probes, the active electrode assembly comprising at least one active electrode element operable to supply an electrical potential of a second polarity different than the first polarity, the active electrode assembly operable to provide an electrically conductive path between a second biological interface and the active electrode element when the probes are placed proximate the second biological interface, where the first and the second biological interfaces are each part of the biological entity, and wherein at least some of the active agent is positioned between the at least one active electrode element and an exterior of the each of the probes.

In another aspect, an iontophoresis device to delivery an active agent to a biological entity by forming a circuit path from a power source via two different portions of the biological entity includes: a handle having a perimeter sized and configured to be grasped, a counter electrode assembly at least partially received in the handle portion of the housing, the counter electrode assembly comprising a counter electrode element operable to supply an electrical potential of a first polarity from a power source, and at least one active electrode assembly including a plurality of probes, at least one active electrode element, and at least one active agent reservoir, each of the plurality of probes extending from the handle and having an exterior that is distinct from one another, the at least one active agent reservoir being positioned between the at least one active electrode element and the exterior the probes, the at least one active electrode element operable to supply an electrical potential of a second polarity, opposite to the first polarity to the active agent reservoir such that at least some active agent is driven from the active agent reservoir through the exterior of the probes in response to the supply of the electrical potential of the second polarity.

In a yet another aspect an iontophoresis device to delivery an active agent to a biological entity by forming a circuit path from a power source via two different portions of the biological entity includes a handle having a perimeter sized and configured to be grasped, a counter electrode assembly at least partially received in the handle portion of the housing, the counter electrode assembly comprising a counter electrode element operable to supply an electrical potential of a first polarity from a power source, and at least one active electrode assembly including a plurality of probe shaped active agent reservoirs each extending from the handle and having an exterior that is distinct from one another, and at least one active electrode element, the at least one active electrode element operable to supply an electrical potential of a second polarity, opposite to the first polarity to the active agent reservoir such that at least some active agent is driven from the active agent reservoir in response to the supply of the electrical potential of the second polarity.

In still another aspect an active agent delivery device to delivery an active agent to a biological entity includes: a power source; probe means selectively positionable proximate a tooth of the biological entity for actively delivering an active agent thereto via an active current path to the power source; and handle means selectively grippable by the biological entity for forming a return current path to the power source via the biological entity.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a schematic diagram of a handheld active agent delivery device being used to delivery active agents to a biological entity according to one illustrated embodiment where the handheld active agent delivery device includes a handle portion configured to be grasped by a first biological interface and a probe to contact a second biological interface.

FIG. 2 is a block diagram of an active electrode assembly of the handheld active agent delivery device positioned on a first portion or biological interface of the biological entity and a counter electrode assembly positioned on a second portion or biological interface of the biological entity to complete an electrical path though the biological entity, according to one illustrated embodiment.

FIG. 3 a partial cross sectional view of the probe of the handheld active agent delivery device, including a receptacle for removably receiving an active agent insert, according to one illustrated embodiment.

FIG. 4 is a cross sectional view of an active agent insert including a active agent reservoir and an outermost ion selective membrane, according to one illustrated embodiment.

FIG. 5 is a cross sectional view of an active agent insert including an active agent impregnated outermost ion selective membrane which may take the form of an ion exchange membrane, according to one illustrated embodiment.

FIG. 6 a partial cross sectional view of the probe of the handheld active agent delivery device, including a receptacle for removably receiving an active agent insert, according to another illustrated embodiment.

FIG. 7 is a cross sectional view of an active agent insert including an outermost ion selective membrane in the form of a bipolar membrane, having an active agent impregnated in an outermost portion, according to one illustrated embodiment.

FIG. 8 is an isometric diagram of a handheld active agent delivery device having an ergonomic handle portion, according to another illustrated embodiment.

FIG. 9 is an isometric diagram of a handheld active agent delivery device having a plurality of probes extending at an angle from the handle portion, according to another illustrated embodiment.

FIG. 10 is a schematic diagram of a portion of a handheld active agent delivery device having a plurality of probes sharing a common active electrode element, according to another illustrated embodiment.

FIG. 11 is a schematic diagram of a portion of a handheld active agent delivery device having a plurality of probes and sharing a common active agent reservoir and active electrode element, according to yet another illustrated embodiment.

FIG. 12 is a schematic diagram of a portion of a handheld active agent delivery device having a plurality of probes and sharing a common active agent reservoir, electrolyte reservoir, inner ion selective membrane and active electrode element, according to yet another illustrated embodiment.

FIG. 13 is a schematic, partially broken diagram of a portion of a handheld active agent delivery device having a plurality of probes, each of which includes a respective ion exchange membrane and active electrode element, according to still another illustrated embodiment.

### DETAILED DESCRIPTION OF THE INVENTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with controllers including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used herein and in the claims, the term “membrane” means a layer, barrier or material, which may, or may not be permeable. Unless specified otherwise, membranes may take the form a solid, liquid or gel, and may or may not have a distinct lattice or cross-linked structure.

As used herein and in the claims, the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein and in the claims, the term “charge selective membrane” means a membrane which substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. Examples of commercially available cation exchange membranes include those available under the designators NEOSEPTA, CM-1, CM-2, CMX, CMS, and CMB from Tokuyama Co., Ltd. Examples of commercially available anion exchange membranes include those available under the designators NEOSEPTA, AM-1, AM-3, AMX, AHA, ACH and ACS also from Tokuyama Co., Ltd.

As used herein and in the claims, the term bipolar membrane means a membrane that has a first portion that is selective to ions of one polarity or charge and a second portion that is selective to ions of the opposite polarity or charge as the first portion. Unless specified otherwise, a bipolar membrane may take the form of a unitary or monolithic membrane structure or may take the form of a multiple membrane structure. The unitary membrane structure may having a first portion including cation ion exchange material or groups and a second portion opposed to the first portion, including anion ion exchange material or groups. The multiple membrane structure may be formed by a cation exchange membrane attached or coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that substantially selective based on a size or molecular weight of the ion. Thus, a semi-permeable membrane substantially passes ions of a first molecular weight or size, while substantially blocking passage of ions of a second molecular weight or size, greater than the first molecular weight or size.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

A used herein and in the claims, the term “reservoir” means any form of mechanism to retain an element or compound in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

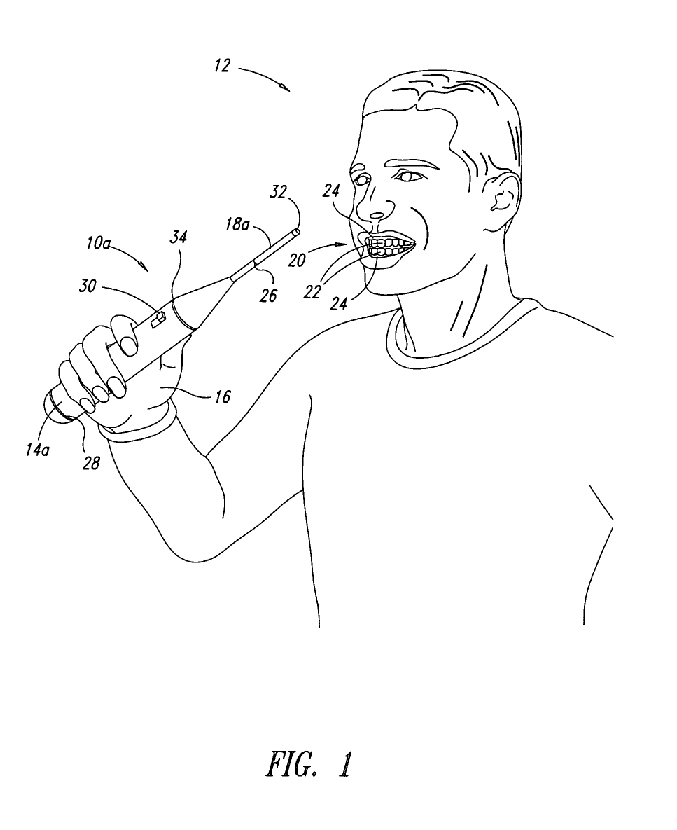

FIG. 1 shows a handheld active agent delivery device 10a being used to delivery active agents to a biological entity 12 according to one illustrated embodiment.

The handheld active agent delivery device 10a includes a handle portion 14a configured to be grasped by a first biological interface 16 and a probe 18a configured to be easily positioned to contact a second biological interface 20. In the embodiment illustrated in FIG. 1, the first biological interface 16 takes the form of all or a portion of a hand that grasps the handle portion 14a of the active agent delivery device 10a, while the second biological interface 20 takes the form of all or a portion of a mouth including a tooth 22, gum 24 or other tissue in a mouth of the biological entity 12.

As illustrated, the handle portion 14a may be sized, dimensioned, shaped or otherwise configured to be easily grasped by the first biological interface 16. Also as illustrated, the probe 18a is sized, dimension, shaped or otherwise configured to be easily positioned in contact with the second biological interface 20. For example, the probe 18a may be elongated and/or have a smaller circumference 26 than a circumference 28 of the handle portion 14a. This may, for example, allow the probe 18a to be positioned in the mouth, adjacent one of the teeth 22, a portion of the gums 24 or proximate some other tissue. Some embodiments of the active agent delivery device 10a described herein may be particularly suited for delivering an active agent to, or proximate, one or more teeth 22. Such may delivery a desensitizing active agent, for example strontium or strontium chloride, to desensitize a tooth 22 or portion of a tooth such as a nerve.

Alternatively, or additionally, the active agent delivery device 10a may be used to deliver small amounts of fluoride under low power to the surface of a tooth 22. This may be especially helpful in pediatric applications, as children are much more susceptible to fluorosis. Fluorosis occurs when too much fluoride is administered (e.g., with rinses), and can harm the teeth 22 and even cause bone problems in excessive amounts. Some toothpastes formulated for children omit fluoride, since children tend to ingest the fluoride which may sometimes cause toxicity. Direct and controlled application to the surface of a tooth 22 would provide the appropriate amount for strengthening the enamel and reduce the occurrence of fluorosis.

Alternatively, or additionally, the active agent delivery device 10a may be used to deliver an anesthetic, for example lidocaine. Such may be used to temporarily alleviate existing pain and/or may be used prior to an injection via a traditional needle and syringe to alleviate pain resulting from the injection.

The handheld active agent delivery device 10a may include a switch 30 accessible from an exterior of the handheld active agent delivery device 10a to allow a user to turn the device ON. The handheld active agent delivery device 10a may include a timer to automatically turn the device OFF after a period of time, which may, or may not be a user configurable time period. Alternatively, or additionally, the switch 30 may also allow the user to turn OFF the handheld active agent delivery device 10a.

The handheld active agent delivery device 10a may further include an active agent insert 32, that allows active agent to be loaded into the delivery device 10a, advantageously allowing most of the device 10a to be reusable.

A housing 34 of the handheld active agent delivery device 10a may be sealed and capable of withstanding sterilization processes, for example high temperatures and/or sanitizing chemical agents. While the active agent insert 32 may be removably received by the probe 18a, in some embodiments the entire probe 18a is removable and constitutes the active agent insert 32.

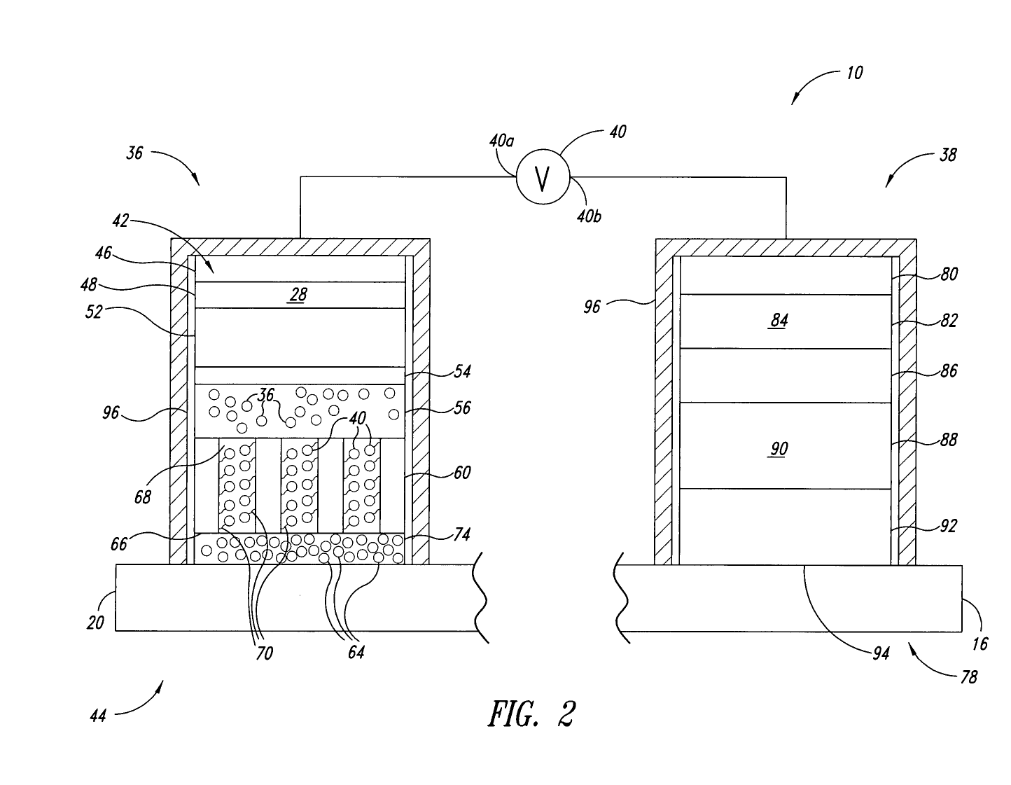

FIG. 2 schematically illustrates a generic version of the handheld active agent delivery devices otherwise described herein, and referred generically as iontophoresis device 10.

The iontophoresis device 10 comprises an active electrode assembly 36 positioned on or proximate a second biological interface 20, and counter assembly 38 positioned proximate a second biological interface 16. Each electrode assembly 36, 38 is electrically coupled to a power source 40 and operable to supply an active agent to the second biological interface 20 via iontophoresis, according to one illustrated embodiment. As noted above, the first and/or the second biological interfaces 16, 20, may take a variety of forms, for example a portion of skin, mucous membrane, tooth, gum other tissue. In the illustrated embodiment, the first biological interface 16 may take the form of all or a portion of the hand (FIG. 1), while the second biological interface 20 may take the form all or a portion of a tooth 22, gum 24, or other tissue in a mouth.

In the illustrated embodiment, the active electrode assembly 26 comprises, from an interior 42 to an exterior 44 of the active electrode assembly 36, an active electrode element 46, an electrolyte reservoir 48 storing an electrolyte 50, an inner ion selective membrane 52, an optional inner sealing liner 54, an inner active agent reservoir 56 storing active agent 58, an outermost ion selective membrane 60 that caches additional active agent 62, and further active agent 64 carried by an outer surface 66 of the outermost ion selective membrane 60. Many of these elements or structures are optional. Each of the above elements or structures are be discussed in detail below.

The active electrode element 46 is coupled to a first pole 40a of the power source 40 and positioned in the active electrode assembly 36 to apply an electromotive force or current to transport active agent 58, 62, 64 via various other components of the active electrode assembly 36. The active electrode element 46 may take a variety of forms. For example, the active electrode element 46 may include a sacrificial element, for example a chemical compound or amalgam including silver (Ag) or silver chloride (AgCl). Such compounds or amalgams typically employ one or more heavy metals, for example lead (Pb), which may present issues with regard manufacturing, storage, use and/or disposal. Consequently, some embodiments may advantageously employ a carbon-based active electrode element 46. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004.

The electrolyte reservoir 48 may take a variety of forms including any structure capable of retaining electrolyte 50, and in some embodiments may even be the electrolyte 50 itself, for example, where the electrolyte 50 is in a gel, semi-solid or solid form. For example, the electrolyte reservoir 48 may take the form of a pouch or other receptacle, a membrane with pores, cavities or interstices, particularly where the electrolyte 50 is a liquid.

The electrolyte 50 may provide ions or donate charges to prevent or inhibit the formation of gas bubbles (e.g., hydrogen) on the active electrode element 46 in order to enhance efficiency and/or increase delivery rates. This elimination or reduction in electrolysis may in turn inhibit or reduce the formation of acids and/or bases (e.g., H⁺ ions, OH⁻ ions), that would otherwise present possible disadvantages such as reduced efficiency, reduced transfer rate, and/or possible irritation of the biological interface 20. As discussed further below, in some embodiments the electrolyte 50 may provide or donate ions to substitute for the active agent, for example and without limitation by theory substituting for the active agent 62 cached in the outermost ion selective membrane 60. Such may facilitate transfer of the active agent 62 to the biological interface 20, for example, increasing and/or stabilizing delivery rates. A suitable electrolyte may take the form of a solution of 0.5M disodium fumarate: 0.5M Poly acrylic acid (5:1).

The inner ion selective membrane 52 is generally positioned to separate the electrolyte 50 and the inner active agent reservoir 56. The inner ion selective membrane 52 may take the form of a charge selective membrane. For example, where the active agent 58, 62, 64 comprises a cationic active agent, the inner ion selective membrane 52 may take the form of an anion exchange membrane, selective to substantially pass anions and substantially block cations. Also, for example, where the active agent 58, 62, 64 comprises an anionic active agent, the inner ion selective membrane 52 may take the form of a cationic exchange membrane, selective to substantially pass cations and substantially block anions. The inner ion selective membrane 52 may advantageously prevent transfer of undesirable elements or compounds between the electrolyte 50 and the active agents 58, 62, 64. For example, the inner ion selective membrane 52 may prevent or inhibit the transfer of hydrogen (H⁺) or sodium (Na⁺) ions from the electrolyte 50, which may increase the transfer rate and/or biological compatibility of the iontophoresis device 10.

The optional inner sealing liner 54 separates the active agent 58, 62, 64 from the electrolyte 50 and is selectively removable. The inner sealing liner 54 may advantageously prevent migration or diffusion between the active agent 58, 62, 64 and the electrolyte 50, for example, during storage.

The inner active agent reservoir 56 is generally positioned between the inner ion selective membrane 52 and the outermost ion selective membrane 60. The inner active agent reservoir 56 may take a variety of forms including any structure capable of temporarily retaining active agent 58, and in some embodiments may even be the active agent 58 itself, for example, where the active agent 58 is in a gel, semi-solid or solid form. For example, the inner active agent reservoir 56 may take the form of a pouch or other receptacle, a membrane with pores, cavities or interstices, particularly where the active agent 58 is a liquid. The inner active agent reservoir 56 may advantageously allow larger doses of the active agent 58 to be loaded in the active electrode assembly 36.

The outermost ion selective membrane 60 is positioned generally opposed across the active electrode assembly 36 from the active electrode element 46. The outermost ion selective membrane 60 may, as in the embodiment illustrated in FIG. 2, take the form of an ion exchange membrane, pores 68 (only one called out in FIG. 2 for sake of clarity of illustration) of the ion selective membrane 60 including ion exchange material or groups 70 (only three called out in FIG. 2 for sake of clarity of illustration). Under the influence of an electromotive force or current, the ion exchange material or groups 70 selectively substantially passes ions of the same polarity as active agent 58, 62, while substantially blocking ions of the opposite polarity. Thus, the outermost ion exchange membrane 60 is charge selective. Where the active agent 58, 62, 64 is a cation (e.g., strontium, lidocaine), the outermost ion selective membrane 60 may take the form of a cation exchange membrane. Alternatively, where the active agent 58, 62, 64 is an anion (e.g., fluoride), the outermost ion selective membrane 60 may take the form of an anion exchange membrane.

The outermost ion selective membrane 60 may advantageously cache active agent 62. In particular, the ion exchange groups or material 70 temporarily retains ions of the same polarity as the polarity of the active agent in the absence of electromotive force or current and, without being limited by theory, substantially releases those ions when replaced with substitutive ions of like polarity or charge under the influence of an electromotive force or current.

Alternatively, the outermost ion selective membrane 60 may take the form of semi-permeable or microporous membrane which is selective by size. In some embodiments, such a semi-permeable membrane may advantageously cache active agent 62, for example by employing a removably releasable outer release liner 72 (FIGS. 4, 5 and 7) to retain the active agent 62 until the outer release liner 72 is/are removed prior to use.

The outermost ion selective membrane 60 may be preloaded with the additional active agent 62, such as ionized or ionizable drugs or therapeutic agents and/or polarized or polarizable drugs or therapeutic agents. Where the outermost ion selective membrane 60 is an ion exchange membrane, a substantial amount of active agent 62 may bond to ion exchange groups 70 in the pores, cavities or interstices 68 of the outermost ion selective membrane 60.

Active agent that fails to bond to the ion exchange groups of material 70 may adhere to the outer surface 66 of the outermost ion selective membrane 60 as the further active agent 64. Alternatively, or additionally, the further active agent 64 may be positively deposited on and/or adhered to at least a portion of the outer surface 66 of the outermost ion selective membrane 60, for example, by spraying, flooding, coating, electrostatically, vapor deposition, and/or otherwise. In some embodiments, the further active agent 64 may sufficiently cover the outer surface 66 and/or be of sufficient thickness so as to form a distinct layer 74. In other embodiments, the further active agent 64 may not be sufficient in volume, thickness or coverage as to constitute a layer in a conventional sense of such term.

The active agent 64 may be deposited in a variety of highly concentrated forms such as, for example, solid form, nearly saturated solution form or gel form. If in solid form, a source of hydration may be provided, either integrated into the active electrode assembly 36, or applied from the exterior thereof just prior to use.

In some embodiments, the active agent 58, additional active agent 62, and/or further active agent 64 may be identical or similar compositions or elements. In other embodiments, the active agent 58, additional active agent 62, and/or further active agent 64 may be different compositions or elements from one another. Thus, a first type of active agent may be stored in the inner active agent reservoir 56, while a second type of active agent may be cached in the outermost ion selective membrane 60. In such an embodiment, either the first type or the second type of active agent may be deposited on the outer surface 66 of the outermost ion selective membrane 60 as the further active agent 64. Alternatively, a mix of the first and the second types of active agent may be deposited on the outer surface 66 of the outermost ion selective membrane 60 as the further active agent 64. As a further alternative, a third type of active agent composition or element may be deposited on the outer surface 66 of the outermost ion selective membrane 60 as the further active agent 64. In another embodiment, a first type of active agent may be stored in the inner active agent reservoir 56 as the active agent 58 and cached in the outermost ion selective membrane 60 as the additional active agent 62, while a second type of active agent may be deposited on the outer surface 66 of the outermost ion selective membrane 60 as the further active agent 64. Typically, in embodiments where one or more different active agents are employed, the active agents 58, 62, 64 will all be of common polarity to prevent the active agents 58, 62, 64 from competing with one another. Other combinations are possible.

In the illustrated embodiment, the counter electrode assembly 38 comprises, from an interior 76 to an exterior 78 of the counter electrode assembly 38: a counter electrode element 80, an electrolyte reservoir 82 storing an electrolyte 84, an inner ion selective membrane 86, an optional inner sealing liner (not illustrated), a buffer reservoir 88 with a buffer agent 90; and an outermost ion selective membrane 92 having an outer surface 94. Many of these structures and/or substances are similar to those of the active electrode assembly 36, although may carry the opposite polarity. For example, the counter electrode element is electrically coupled to pole 40b of the power source 40. Also for example, where the active electrode assembly 36 employs a CEM, the counter electrode may employ an AEM. Only significant differences are discussed below.

The buffer reservoir 88 may supply ions or charge to balance the ions transferred through the outermost counter ion selective membrane 92 from the biological interface 16. Consequently, the buffer agent 90 may, for example, comprise a salt (e.g., NaCl). The buffer agent 90 may be temporarily retained by a buffer reservoir 88. The buffer reservoir 88 may take a variety of forms capable of temporarily retaining the buffer agent 90. For example, the buffer reservoir 88 may take the form of a membrane forming a cavity, a porous membrane or a gel.

An interface coupling medium (not shown) may be employed between the active electrode assembly 36 and the biological interface 20. The interface coupling medium may, for example, take the form of an adhesive and/or gel. The gel may, for example, take the form of a hydrating gel.

The power source 40 may take the form of one or more chemical battery cells, super- or ultra-capacitors, or fuel cells. The power source 40 may, for example, provide a voltage of 12.8V DC, with tolerance of 0.8V DC, and a current of 0.3 mA. The power source 40 may be selectively electrically coupled to the active and counter electrode assemblies 36, 38 via a control circuit, for example, via carbon fiber ribbons. The iontophoresis device 10 may include discrete and/or integrated circuit elements to control the voltage, current and/or power delivered to the electrode assemblies 36, 38. For example, the iontophoresis device 10 may include a diode to provide a constant current to the electrode assemblies 36, 38.

As suggested above, the active agent 58, 62, 64 may take the form of a cationic or an anionic drug or other therapeutic agent. Consequently, the poles or terminals 40a, 40b of the power source 40 may be reversed. Likewise, the selectivity of the outermost ion selective membrane 60 and inner ion selective membranes 54 may be reversed.

The iontophoresis device 10 may further comprise an inert molding material 96 adjacent exposed sides of the various other structures forming the active and counter electrode assemblies 36, 38. The molding material 96 may advantageously provide environmental protection to the various structures of the active and counter electrode assemblies 36, 38. Molding material 96 may form a slot or opening (not shown) on one of the exposed sides through which the tab (not shown) extends to allow for the removal of inner sealing liner 54 prior to use. Enveloping the active and counter electrode assemblies 36, 38 may be a housing material (not shown) The housing material may also form a slot or opening (not shown) positioned aligned with the slot or opening in molding material 96 through which the tab extends to allow for the removal of inner sealing liner 54 prior to use of the iontophoresis device 10, as described below.

Immediately prior to use, the iontophoresis device 10 is prepared by withdrawing the inner sealing liner 54 and removing the outer release liners 72 (FIGS. 4, 5 and 7). As described above, the inner sealing liner 54 may be withdrawn by pulling on a tab. The outer release liners 72 may be pulled off in a similar fashion to remove release liners from pressure sensitive labels and the like.

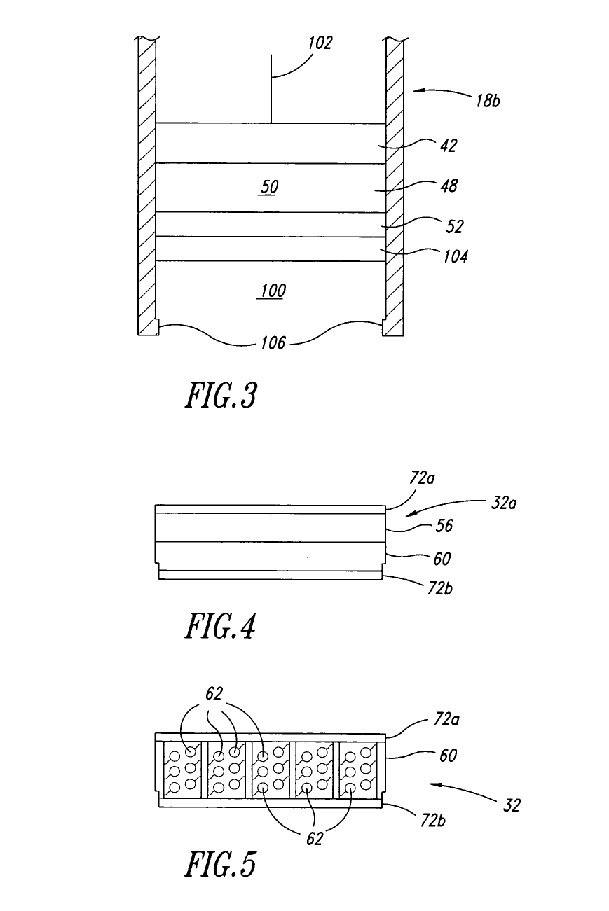

FIG. 3 shows a portion of a probe 18b, illustrating a receptacle 100 to receive the active agent insert 32, according to one illustrated embodiment.

The probe 18b may include some or all of the membranes, reservoirs and other structures of the active electrode assembly 36 discussed above. For example, the probe 18b may, for example, include the active electrode element 46 coupled to the power source 40 via an electrically conductive current path such as a lead 102. The probe 18b may also, for example, include the electrolyte reservoir 48 and/or electrolyte 50, an inner ion selective membrane 52. The probe 18b may further, for example, include a spacer such as a spacer or porous (e.g., nonselective) membrane 104 to space the inner ion selective membrane 52 from the outermost ion selective membrane 60. Such may advantageously reduce the occurrence of hydrolysis of water. The remainder of the active electrode assembly 36 may be located in the active agent insert 32.

The probe 18b may from a detent 106 or other retaining mechanism for releasably or removably securing the active agent insert 32 in the receptacle 100. Alternatively, the active agent insert 32 may be sized and dimensions to create a friction fit with the wall of the receptacle 100.

FIG. 4 shows an active agent insert 32a, according to one illustrated embodiment, usable with the probe 18b illustrated in FIG. 3.

The active agent insert 32a may, for example, include an active agent reservoir 56 storing active agent 58 (FIG. 2). The active agent 58 may, for example, take the for of strontium, strontium chloride or some other strontium compound, useful for desensitizing teeth 22 (FIG. 1). The active agent insert 32a may also, for example, include an outermost ion selective membrane 60, for example an outermost ion exchange membrane. The outermost ion selective membrane 60 may be impregnated or otherwise cache additional active agent 62 (FIG. 2), and may include further active agent 64 (FIG. 2) carried on an outermost surface 66 (FIG. 2) thereof.

An inner release liner 72a may generally be positioned overlying or covering the active agent reservoir 56. An outer release liner 72b may generally be positioned overlying or covering the further active agent 64 carried by the outer surface 66 of the outermost ion selective membrane 60. The inner release liner 72a may protect the active agent reservoir 56 during storage, prior to application of an electromotive force or current. The outer release liner 72b may protect the further active agent 64 and/or outermost ion selective membrane 60 during storage, prior to application of an electromotive force or current. The inner and/or outer release liners 72a, 72b may be a selectively releasable liner made of waterproof material, such as release liners commonly associated with pressure sensitive adhesives. Note that the inner and outer release liners 72a, 72b are shown removed in FIG. 2.

FIG. 5 shows an active agent insert 32b, according to one illustrated embodiment, usable with the probe 18b illustrated in FIG. 3.

The active agent insert 32b may, for example, include an outermost ion selective membrane 60, for example an outermost ion exchange membrane. The outermost ion selective membrane 60 may be impregnated or otherwise cache active agent 62, and may include further active agent 64 (FIG. 2) carried on an outermost surface 66 (FIG. 2) thereof. The active agent 62, 62 may, for example, take the form of strontium, strontium chloride or some other strontium compound, useful for desensitizing teeth 22 (FIG. 1).

The active agent insert 32b may also, for example, include an inner release liner 72a and an outer release liner 72b, each of the release liners 72a, 72b generally be positioned overlying or covering a respective face of the outermost ion selective membrane 60. The inner and outer release liners 72a, 72b may protect the outermost ion selective membrane 60 during storage, prior to application of an electromotive force or current.

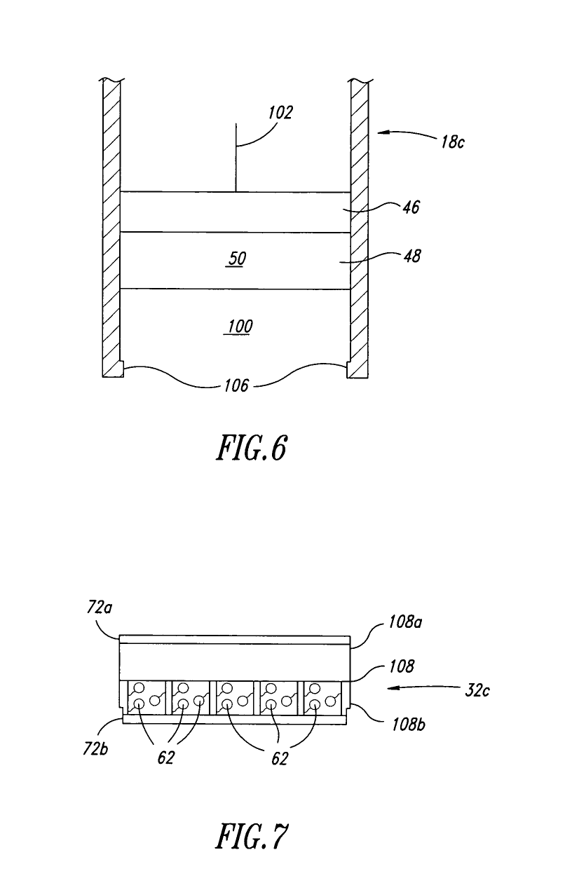

FIG. 6 shows a portion of the probe 18c, illustrating a receptacle 100 to receive an active agent insert 32, according to one illustrated embodiment.

The probe 18c may include some or all of the membranes, reservoirs and other structures of the active electrode assembly 36 discussed above. For example, the probe 18c may, for example, include the active electrode element 42 coupled to the power source 40 via an electrically conductive current path such as a lead 102. The probe 18c may also, for example, include the electrolyte reservoir 48 and/or electrolyte 50. The remainder of the active electrode assembly 36 may be located in the active agent insert 32, or omitted altogether.

The probe 18c may have a detent 106 or other retaining mechanism for releasably or removably securing the active agent insert 32 in the receptacle 100. Alternatively, the active agent insert 32 may be sized and dimensions to create a friction fit with the wall of the receptacle 100.

FIG. 7 shows an active agent insert 32c, according to one illustrated embodiment, usable with the probe 18c (FIG. 6).

The active agent insert 32c may, for example, include a bipolar membrane 108. An inner portion 108a of the bipolar membrane 108 may take the form of an ion exchange membrane that is permselective to ions of an opposite polarity as the polarity of the active agent, while an outer portion 108b may take the form of an ion exchange membrane that is permselective to ions of a same or like polarity as the polarity of the active agent. The bipolar membrane 108 may be formed from separate films, or may be a single film membrane with appropriate ion exchange materials or groups deposited or distributed into the respective inner and outer portions 108a, 108b.

The outer portion 108b of the bipolar membrane 108 may be impregnated or otherwise cache active agent 62. Further active agent 64 (FIG. 2) may be carried on an outermost surface 66 (FIG. 2) thereof. The active agent 62, 64 may, for example, take the for of strontium, strontium chloride or some other strontium compound, useful for desensitizing teeth 22 (FIG. 1).

The active agent insert 32c may also, for example, include an inner release liner 72a and an outer release liner 72b, each of the release liners 72a, 72b generally be positioned overlying or covering a respective face of the bipolar membrane 108. The inner and outer release liners 72a, 72b may protect the bipolar membrane 108 and active agent 62, 64 during storage, prior to application of an electromotive force or current.

FIG. 8 shows a handheld active agent delivery device 10b according to another illustrated embodiment. The handheld active agent delivery device 10b has an ergonomically configured handle portion 14b, having a number of ridges 110 and valleys 112 for comfortably accommodating the digits (e.g., fingers) of a hand (FIG. 1). Other ergonomic configurations are possible. The handheld active agent delivery device 10b may also include a cord or wire 114 to couple the handheld active agent delivery device 10b to an external power source 16 and/or controller (not shown).

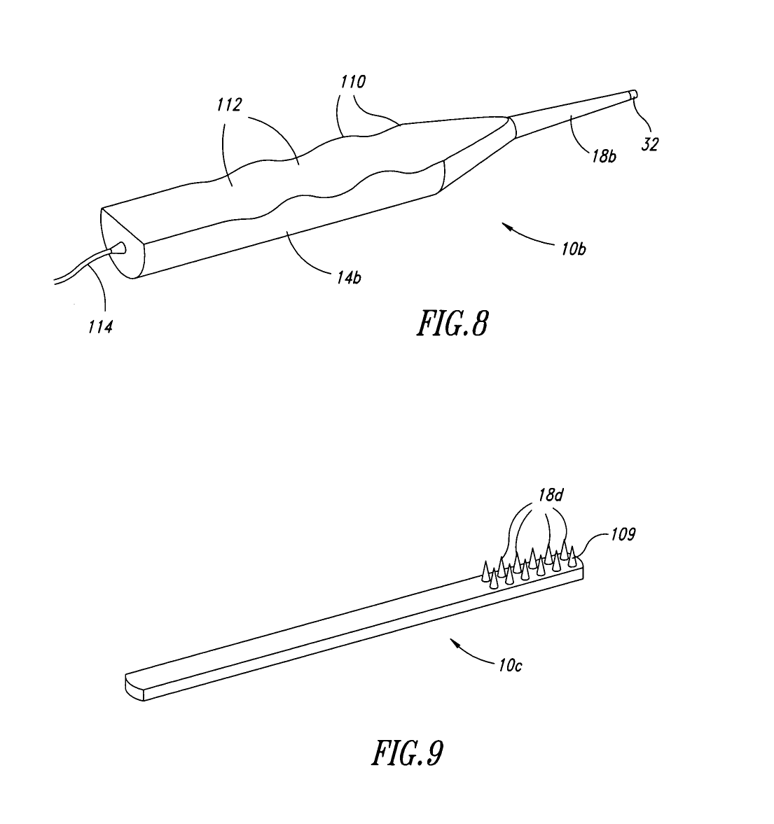

FIG. 9 shows a handheld active agent delivery device 10c according to a further illustrated embodiment. The handheld active agent delivery device 10c has a flattened handle portion 14c, with a plurality of probes 18d extending upwardly at an angle (e.g., 90 degrees) therefrom, and may resemble a common tooth brush. Each of the probes is distinct from one another. Each of the probes has an exterior 109 and an interior 111 (FIG. 13). While illustrated generally as a right angle, the probes 18d may extend from the handle portion 14c at other angles, to accommodate the particular biological structure of the intended use, and various ones of the probes 18d may extend at different angles from one another. While illustrated as having a plurality of probes 18d, other embodiments may include a greater or fewer number of probes 18d extending from the handle portion 14c. For example, one embodiment may include a single probe 18d extending at a right angle, an acute angle or an obtuse angle from the handle portion 14c.

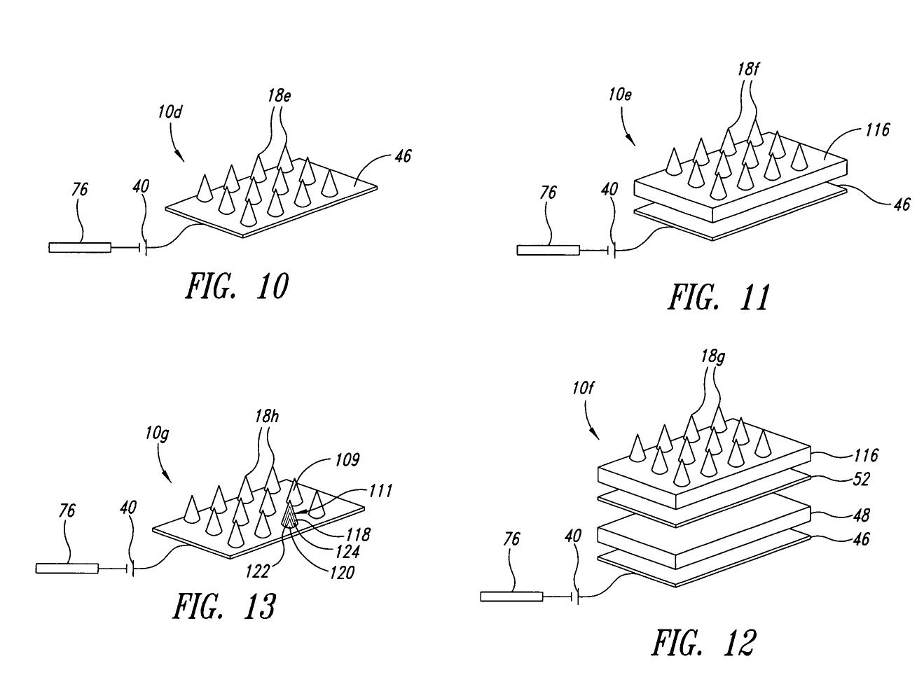

FIG. 10 shows a portion of a handheld active agent delivery device 10d, according to another illustrated embodiment. The handheld active agent delivery device 10d includes a plurality of probes 18e which may take the form of probe shaped active agent reservoirs, each of which is capable of temporarily storing active agent 58, 62, 64 (FIG. 2) for delivery to the biological interface 20. The probes 18e may take any of the forms discussed herein, which are suitable as active agent reservoirs for holding active agent 58, 62, 64. For example, the probes 18e may take the form of one or more ion exchange membranes similar to outer ion exchange membrane 60 (FIG. 2). For example, the probes 18e may be formed as individual probe shaped ion exchange membranes on a substrate. Alternatively, the probes 18e may be formed from a monolithic ion exchange membrane, for example via etching or depositioning.

The handheld active agent delivery device 10d also includes at least one active electrode element 46 that is operable to provide an electromotive force of like-polarity as that of the active agent 58, 62, 64, and a counter electrode element 76 that is operable to provide an electromotive force that is opposite that of the active agent. The active and counter electrode elements 46, 76 are electrically coupled to the power source 40, such as one or more battery cells, super- or ultra-capacitors and/or fuel cells. The active electrode element 46 is positioned to provide the electromotive force to two or more of the probes 18e, and thus is common to a plurality of the probes 18e.

FIG. 11 shows a portion of a handheld active agent delivery device 10e, according to yet another illustrated embodiment. The handheld active agent delivery device 10e includes a plurality of probes 18f that extend at an angle from a handle (not illustrated in FIG. 11).

The handheld active agent delivery device 10e also includes at least one active agent reservoir 116. The active agent reservoir 116 may take any of the forms discussed herein, which are suitable for temporarily holding the active agent 58, 62, 64 (FIG. 2). For example, the active agent reservoir 116 may take the form of one or more ion exchange membranes, similar to outer ion exchange membrane 60 (FIG. 2). The active agent reservoir 116 is positioned to provide active agent 58, 62, 64 (FIG. 2) to two or more probes 18f. Thus, the active agent reservoir 116 is common to a plurality of the probes 18f.

The handheld active agent delivery device 10e further includes at least one active electrode element 46 that is operable to provide an electromotive force of like-polarity as that of the active agent, and a counter electrode 76 that is operable to provide an electromotive force that is opposite that of the active agent. The active and counter electrode elements 46, 76 are electrically coupled to a power source 40, such as one or more battery cells, super- or ultra-capacitors and/or fuel cells. The active electrode element 76 is positioned to provide the electromotive force to two or more portions of the active agent reservoir 116 which are in fluid communication with respective ones of two or more of the probes 18f, and thus is common to a plurality of the probes 18f.

FIG. 12 shows a portion of a handheld active agent delivery device 10f, according to yet another illustrated embodiment. The handheld active agent delivery device 10f includes a plurality of probes 18g that extend at an angle from a handle (not illustrated in FIG. 12).

The handheld active agent delivery device 10f also includes at least one active agent reservoir 116. The active agent reservoir 116 may take any of the forms discussed herein, which are suitable for temporarily holding the active agent 58, 62, 64 (FIG. 2). For example, the active agent reservoir 116 may take the form of one or more ion exchange membranes, similar to the outer ion exchange membrane 60 (FIG. 2). The active agent reservoir 116 is positioned to provide active agent 58, 62, 64 to two or more probes 18g. Thus, the active agent reservoir 116 is common to a plurality of the probes 18g.

The handheld active agent delivery device 10f further includes at least one active electrode element 46 that is operable to provide an electromotive force of like-polarity as that of the active agent, and a counter electrode 76 that is operable to provide an electromotive force that is opposite that of the active agent. The active and counter electrode elements 46, 76 are electrically coupled to a power source 40, such as one or more battery cells, fuel cells and/or super- or ultra-capacitors. The active electrode element 46 is positioned to provide the electromotive force to two or more portions of the active agent reservoir 116 which are in fluid communication with respective ones of two or more of the probes 18g, and thus is common to a plurality of the probes 18g.

The handheld active agent delivery device 10f optionally includes at least one electrolyte reservoir 48 positioned between the active agent reservoir 116 and the active electrode element 46. The electrolyte reservoir 48 is capable of storing an electrolyte 50 (FIG. 2), which in some embodiments may be the same substance as the active agent 58, 62, 64 (FIG. 2). The electrolyte reservoir 48 may take any of the forms discussed herein. The benefits of an electrolyte reservoir 48 and an electrolyte 50 have been previously explained and are not repeated here in the interest of brevity.

The handheld active agent delivery device 10f also optionally includes at least one inner ion selective membrane 52 separating the electrolyte reservoir 48 from the active agent reservoir 116. The inner ion selective membrane 52 may take any of the forms discussed herein. The benefits of the inner ion selective membrane 52 have been previously explained and are not repeated here in the interest of brevity.

FIG. 13 shows is a schematic, partially broken diagram of a handheld active agent delivery device 10g, according to still another illustrated embodiment. The handheld active agent delivery device 10g includes a plurality of probes 18h that extend at an angle from a handle (not illustrated in FIG. 13). As illustrated, one of the probes 18h is broken to better show the internal structure thereof.

Each of the probes 18h includes a respective active agent reservoir 118 and active electrode element 120. The active agent reservoirs 118 may take any of the variety of forms discussed herein. The active electrode elements 120 may take any of the variety of forms discussed herein.

Each of the probes 18h may optionally include respective electrolyte reservoir 122 and/or membranes 124. The electrolyte reservoir 122 and/or electrolyte 50 (FIG. 2) may take any of the variety of forms discussed herein. The membranes 124 may take any of the variety of forms discussed herein, including but not limited to porous membranes, semi-permeable membranes, ion selective membranes, ion exchange membranes and/or bipolar membranes.

The above description of illustrated embodiments, including what is described in the Abstract, is not intended to be exhaustive or to limit the claims to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to other agent delivery systems and devices, not necessarily the exemplary iontophoresis active agent system and devices generally described above. For instance, some embodiments may include additional structure. For example, some embodiment may include a control circuit or subsystem to control a voltage, current or power applied to the active and counter electrode elements 36, 38. Also for example, some embodiments may include an interface layer interposed between the outermost active electrode ion selective membrane 60 and the biological interface 20. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240995, herein incorporated in their entirety by reference. Further examples of such hydrogels are disclosed in U.S. Patent applications 2004/166147; 2004/105834; and 2004/247655, herein incorporated in their entirety by reference. Product brand names of various hydrogels and hydrogel sheets include Corplex™ by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd.

During iontophoresis, the electromotive force across the electrode assemblies, as described, leads to a migration of charged active agent molecules, as well as ions and other charged components, through the biological interface into the biological tissue. This migration may lead to an accumulation of active agents, ions, and/or other charged components within the biological tissue beyond the interface. During iontophoresis, in addition to the migration of charged molecules in response to repulsive forces, there is also an electroosmotic flow of solvent (e.g., water) through the electrodes and the biological interface into the tissue. In certain embodiments, the electroosmotic solvent flow enhances migration of both charged and uncharged molecules. Enhanced migration via electroosmotic solvent flow may occur particularly with increasing size of the molecule.

In certain embodiments, the active agent may be a higher molecular weight molecule. In certain aspects, the molecule may be a polar polyelectrolyte. In certain other aspects, the molecule may be lipophilic. In certain embodiments, such molecules may be charged, may have a low net charge, or may be uncharged under the conditions within the active electrode. In certain aspects, such active agents may migrate poorly under the iontophoretic repulsive forces, in contrast to the migration of small more highly charged active agents under the influence of these forces. These higher molecular active agents may thus be carried through the biological interface into the underlying tissues primarily via electroosmotic solvent flow. In certain embodiments, the high molecular weight polyelectrolytic active agents may be proteins, polypeptides or nucleic acids.

The various embodiments discussed above may advantageously employ various microstructures, for example microneedles. Microneedles and microneedle arrays, their manufacture, and use have been described. Microneedles, either individually or in arrays, may be hollow; solid and permeable; solid and semi-permeable; or solid and non-permeable. Solid, non-permeable microneedles may further comprise grooves along their outer surfaces. Microneedle arrays, comprising a plurality of microneedles, may be arranged in a variety of configurations, for example rectangular or circular. Microneedles and microneedle arrays may be manufactured from a variety of materials, including silicon; silicon dioxide; molded plastic materials, including biodegradable or non-biodegradable polymers; ceramics; and metals. Microneedles, either individually or in arrays, may be used to dispense or sample fluids through the hollow apertures, through the solid permeable or semi-permeable materials, or via the external grooves. Microneedle devices are used, for example, to deliver a variety of compounds and compositions to the living body via a biological interface, such as skin or mucous membrane. In certain embodiments, the compounds and active agents may be delivered into or through the biological interface. For example, in delivering compounds or compositions via the skin, the length of the microneedle(s), either individually or in arrays, and/or the depth of insertion may be used to control whether administration of a compound or composition is only into the epidermis, through the epidermis to the dermis, or subcutaneous. In certain embodiments, microneedle devices may be useful for delivery of high-molecular weight compounds and active agents, such as those comprising proteins, peptides and/or nucleic acids, and corresponding compositions thereof. In certain embodiments, for example wherein the fluid is an ionic solution, microneedle(s) or microneedle array(s) can provide electrical continuity between a power source and the tip of the microneedle(s). Microneedle(s) or microneedle array(s) may be used advantageously to deliver or sample compounds or compositions by iontophoretic methods, as disclosed herein. In certain embodiments, for example, a plurality of microneedles in an array may advantageously be formed on an outermost biological interface-contacting surface of an iontophoresis device. Compounds or compositions delivered or sample by such a device may comprise, for example, high-molecular weight molecules or active agents, such as proteins, peptides and/or nucleic acids.

In certain embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; an active agent reservoir having an active agent solution that is in contact with the first electrode member and to which is applied a voltage via the first electrode member; a biological interface contact member, which may be a microneedle array and is placed against the forward surface of the active agent reservoir; and a first cover or container that accommodates these members. The counter electrode assembly includes the following: a second electrode member connected to a negative electrode of the power source; a second electrolyte reservoir that holds an electrolyte that is in contact with the second electrode member and to which voltage is applied via the second electrode member; and a second cover or container that accommodates these members.

In certain other embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; a first electrolyte reservoir having an electrolyte that is in contact with the first electrode member and to which is applied a voltage via the first electrode member; a first anion-exchange membrane that is placed on the forward surface of the first electrolyte reservoir; an active agent reservoir that is placed against the forward surface of the first anion-exchange membrane; a biological interface contacting member, which may be a microneedle array and is placed against the forward surface of the active agent reservoir; and a first cover or container that accommodates these members. The counter electrode assembly includes the following: a second electrode member connected to a negative electrode of the power source; a second electrolyte reservoir having an electrolyte that is in contact with the second electrode member and to which is applied a voltage via the second electrode member; a cation-exchange membrane that is placed on the forward surface of the second electrolyte reservoir; a third electrolyte reservoir that is placed against the forward surface of the cation-exchange membrane and holds an electrolyte to which a voltage is applied from the second electrode member via the second electrolyte reservoir and the cation-exchange membrane; a second anion-exchange membrane placed against the forward surface of the third electrolyte reservoir; and a second cover or container that accommodates these members.

Certain details of microneedle devices, their use and manufacture, are disclosed in U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; 6,939,311; all of which are incorporated herein by reference in their entirety. Some or all of the teaching therein may be applied to microneedle devices, their manufacture, and their use in iontophoretic applications.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: Japanese patent application Serial No. H03-86002, filed Mar. 27, 1991, having Japanese Publication No. H04-297277, issued on Mar. 3, 2000 as Japanese Patent No. 3040517; Japanese patent application Serial No. 11-033076, filed Feb. 10, 1999, having Japanese Publication No. 2000-229128; Japanese patent application Serial No. 11-033765, filed Feb. 12, 1999, having Japanese Publication No. 2000-229129; Japanese patent application Serial No. 11-041415, filed Feb. 19, 1999, having Japanese Publication No. 2000-237326; Japanese patent application Serial No. 11-041416, filed Feb. 19, 1999, having Japanese Publication No. 2000-237327; Japanese patent application Serial No. 11-042752, filed Feb. 22, 1999, having Japanese Publication No. 2000-237328; Japanese patent application Serial No. 11-042753, filed Feb. 22, 1999, having Japanese Publication No. 2000-237329; Japanese patent application Serial No. 11-099008, filed Apr. 6, 1999, having Japanese Publication No. 2000-288098; Japanese patent application Serial No. 11-099009, filed Apr. 6, 1999, having Japanese Publication No. 2000-288097; PCT patent application WO 2002JP4696, filed May 15, 2002, having PCT Publication No. WO03037425; U.S. patent application Ser. No. 10/488,970, filed Mar. 9, 2004; Japanese patent application 2004/317317, filed Oct. 29, 2004; U.S. provisional patent application Ser. No. 60/627,952, filed Nov. 16, 2004; Japanese patent application Serial No. 2004-347814, filed Nov. 30, 2004; Japanese patent application Serial No. 2004-357313, filed Dec. 9, 2004; Japanese patent application Serial No. 2005-027748, filed Feb. 3, 2005; Japanese patent application Serial No. 2005-081220, filed Mar. 22, 2005, and U.S. provisional patent application Ser. No. 60/722,759, filed Sep. 30, 2005.

Aspects of the various embodiments can be modified, if necessary, to employ systems, circuits and concepts of the various patents, applications and publications to provide yet further embodiments. While some embodiments may include all of the membranes, reservoirs and other structures discussed above, other embodiments may omit some of the membranes, reservoirs or other structures. Still other embodiments may employ additional ones of the membranes, reservoirs and structures generally described above. Even further embodiments may omit some of the membranes, reservoirs and structures described above while employing additional ones of the membranes, reservoirs and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

We/I claim:

1. A device for delivering an active agent under the influence a power source to a biological entity, the device comprising:
a handle sized and dimensioned to be grasped;
a plurality of probes extending from the handle;
a counter electrode assembly at least partially positioned in the handle, the counter electrode assembly comprising at least a counter electrode element operable to supply an electrical potential of a first polarity, the counter electrode assembly operable to provide an electrically conductive path between a first biological interface and the counter electrode element when the handle is grasped; and
at least one active electrode assembly positioned proximate the plurality of probes, the active electrode assembly comprising at least one active electrode element operable to supply an electrical potential of a second polarity different than the first polarity, the active electrode assembly operable to provide an electrically conductive path between a second biological interface and the active electrode element when the probes are placed proximate the second biological interface, where the first and the second biological interfaces are each part of the biological entity, and wherein at least some of the active agent is positioned between the at least one active electrode element and an exterior of the each of the probes.

2. The device of claim 1 wherein the active agent comprises strontium.

3. The device of claim 2 wherein the active electrode element comprises silver.

4. The device of claim 2 wherein the active electrode element comprises a compound of silver chloride.

5. The device of claim 1 wherein the active agent comprises strontium chloride.

6. The device of claim 1 wherein the active agent comprises fluoride.

7. The device of claim 1 wherein the active electrode assembly has at least one receptacle formed therein, sized to removably receive the active agent formed as an insert.

8. The device of claim 7, further comprising:
the active agent formed as the insert.

9. The device of claim 8 wherein the insert comprises the active agent in a gel form.

10. The device of claim 1 wherein the active electrode assembly further comprises an outermost ion selective membrane positioned such that the outermost ion exchange membrane is between the active electrode and the first biological interface the when in use, and an electrolyte positioned between the active electrode element and the outermost ion selective membrane.

11. The device of claim 10 wherein the outermost ion selective membrane is an ion exchange membrane permselective to ions of the second polarity; and further comprising:
an inner ion selective membrane positioned between the electrolyte and the outermost ion selective membrane, wherein the inner ion selective membrane is an ion exchange membrane permselective to ions of the first polarity.

12. The device of claim 11, further comprising:
the power source having a first pole and a second pole, the first pole electrically coupled to the counter electrode element and the second pole electrically coupled to the active electrode element.

13. An iontophoresis device to deliver an active agent to a biological entity by forming a circuit path from a power source via two different portions of the biological entity, the iontophoresis device comprising:
a handle having a perimeter sized and configured to be grasped;
a counter electrode assembly at least partially received in the handle portion of the housing, the counter electrode assembly comprising a counter electrode element operable to supply an electrical potential of a first polarity from a power source; and
at least one active electrode assembly including a plurality of probes, at least one active electrode element, and at least one active agent reservoir, each of the plurality of probes extending from the handle and having an exterior that is distinct from one another, the at least one active agent reservoir being positioned between the at least one active electrode element and the exterior the probes, the at least one active electrode element operable to supply an electrical potential of a second polarity, opposite to the first polarity to the active agent reservoir such that at least some active agent is driven from the active agent reservoir through the exterior of the probes in response to the supply of the electrical potential of the second polarity.

14. The iontophoresis device of claim 13 wherein the active electrode element comprises a quantity of silver and wherein the active electrode assembly further comprises: a strontium chloride active agent positioned to be delivered to a first portion of the biological entity when the active electrode element is electrically coupled to a second pole of the power source, the counter electrode is electrically coupled to a first pole of the power source and the counter electrode assembly is positioned at least proximate the second portion of the biological entity.

15. The iontophoresis device of claim 13 wherein the active electrode assembly further comprises: a strontium chloride active agent positioned to be delivered at least proximate a tooth of the biological entity when the active electrode element is electrically coupled to a second pole of the power source, the counter electrode is electrically coupled to a first pole of the power source and the counter electrode assembly is positioned a hand of the biological entity while the handle portion is grasped by the biological entity.

16. The iontophoresis device of claim 15 wherein the strontium chloride active agent is stored in at least one of the active agent reservoirs, selectively insertable in the active electrode assembly.

17. The iontophoresis device of claim 15 wherein the strontium chloride active agent is formed as a number of gel type active agent reservoirs, selectively insertable in the active electrode assembly.

18. The iontophoresis device of claim 13 wherein the active electrode assembly further comprises: an outermost ion selective membrane positioned in the probe to be proximate one of the portions of the biological entity when in use, and an active agent cached in the outermost ion selective membrane.

19. The iontophoresis device of claim 18 wherein the outermost ion selective membrane is an ion exchange membrane substantially selectively permeable by ions of the second polarity and substantially non-permeable to ions of the first polarity.

20. The iontophoresis device of claim 13 wherein the active electrode assembly further comprises: a fluoride active agent positioned to be delivered at least proximate a tooth of the biological entity when the active electrode element is electrically coupled to a second pole of the power source, the counter electrode is electrically coupled to a first pole of the power source and the counter electrode assembly is positioned a hand of the biological entity while the handle portion is grasped by the biological entity.

21. The iontophoresis device of claim 13 wherein each of the probes has a perimeter smaller than the perimeter of the handle.

22. An iontophoresis device to deliver an active agent to a biological entity by forming a circuit path from a power source via two different portions of the biological entity, the iontophoresis device comprising:
a handle having a perimeter sized and configured to be grasped;
a counter electrode assembly at least partially received in the handle portion of the housing, the counter electrode assembly comprising a counter electrode element operable to supply an electrical potential of a first polarity from a power source; and
at least one active electrode assembly including a plurality of probe shaped active agent reservoirs each extending from the handle and having an exterior that is distinct from one another, and at least one active electrode element, the at least one active electrode element operable to supply an electrical potential of a second polarity, opposite to the first polarity to the active agent reservoir such that at least some active agent is driven from the active agent reservoir in response to the supply of the electrical potential of the second polarity.

23. The iontophoresis device of claim 22 wherein the active electrode element comprises a quantity of silver and wherein the active electrode assembly further comprises: a strontium chloride active agent positioned to be delivered to a first portion of the biological entity when the active electrode element is electrically coupled to a second pole of the power source, the counter electrode is electrically coupled to a first pole of the power source and the counter electrode assembly is positioned at least proximate the second portion of the biological entity.

24. The iontophoresis device of claim 22 wherein the active electrode assembly further comprises: a strontium chloride active agent positioned to be delivered at least proximate a tooth of the biological entity when the active electrode element is electrically coupled to a second pole of the power source, the counter electrode is electrically coupled to a first pole of the power source and the counter electrode assembly is positioned a hand of the biological entity while the handle portion is grasped by the biological entity.

25. The iontophoresis device of claim 24 wherein the strontium chloride active agent is stored in a number of probe shaped active agent reservoirs, selectively insertable in the active electrode assembly.

26. The iontophoresis device of claim 24 wherein the strontium chloride active agent is formed as a number of probe shaped gel type active agent reservoirs, selectively insertable in the active electrode assembly.

27. The iontophoresis device of claim 22 wherein probed shaped active agent reservoirs include an outermost ion selective membrane positioned to be proximate one of the portions of the biological entity when in use, and an active agent cached in the outermost ion selective membrane.

28. The iontophoresis device of claim 27 wherein the outermost ion selective membrane is an ion exchange membrane substantially selectively permeable by ions of the second polarity and substantially non-permeable to ions of the first polarity.

29. The iontophoresis device of claim 22 wherein the active electrode assembly further comprises: a fluoride active agent positioned to be delivered at least proximate a tooth of the biological entity when the active electrode element is electrically coupled to a second pole of the power source, the counter electrode is electrically coupled to a first pole of the power source and the counter electrode assembly is positioned a hand of the biological entity while the handle portion is grasped by the biological entity.

30. An active agent delivery device to deliver an active agent to a biological entity, the active agent delivery device comprising:
a power source;
a plurality of probe means selectively positionable proximate a tooth of the biological entity for actively delivering an active agent thereto via an active current path to the power source; and
handle means selectively grippable by the biological entity for forming a return current path to the power source via the biological entity.

31. The active agent delivery device of claim 30 wherein each of the probe means comprises a probe shaped housing receiving an active electrode element electrically coupled to the power source, an electrolyte proximate the active electrode element, an active agent, and an outermost ion selective membrane exposed through the probe shaped housing to an exterior thereof.

32. The active agent delivery device of claim 31 wherein the active agent is temporarily cached in the outermost ion selective membrane.

33. The active agent delivery device of claim 31 wherein the active agent is formed as a replaceable insert comprising a quantity of strontium.

34. The active agent delivery device of claim 31 wherein the active agent is formed as a replaceable insert comprising a quantity of fluoride.

35. The active agent delivery device of claim 30 wherein each of the probe means comprises a respective probe shaped active agent reservoir.

36. The active agent delivery device of claim 35 wherein each of the probe means includes a respective an electrolyte reservoir.

37. The active agent delivery device of claim 35 wherein each of the probe means includes a respective ion selective membrane.

38. The active agent delivery device of claim 35 wherein each of the probe means share a common active electrode element electrically coupled to the power source.

39. The active agent delivery device of claim 35 wherein each of the probe means share a common electrolyte reservoir.

40. The active agent delivery device of claim 35 wherein each of the probe means share a common ion selective membrane.

41. The active agent delivery device of claim 30 wherein the handle means comprises a handle shaped housing receiving a counter electrode element electrically coupled to the power source, an electrolyte proximate the counter electrode element, and an outermost ion selective membrane exposed through the handle shaped housing to an exterior thereof.
